(19) United States
(12) Patent Application Publication
(10) Pub. No.: US 2005/0228380 A1
(43) Pub. Date: Oct. 13, 2005

(54) Instruments and methods for minimally invasive spine surgery

(75) Inventors: Bradley Moore, Barrington, RI (US); Ronald Naughton, Tiverton, RI (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: DePuy Spine Inc., Raynham, MA (US)

(21) Appl. No.: 10/821,284

(22) Filed: Apr. 9, 2004

(51) Int. Cl. A61B 17/56

(52) U.S. Cl. 606/61

(57) ABSTRACT

A minimally invasive surgical method may include positioning a first anchor and second anchor in a first vertebra and a second vertebra, respectively, through a first incision made on a first side of a patient's spine, percutaneously positioning a third anchor in a third vertebra through a second incision distinct from the first incision, advancing the first end of a fixation element from the first incision subcutaneously to the third anchor, and coupling the fixation element to the first anchor, the second anchor, and the third anchor.

### BACKGROUND

For a number of known reasons, spinal fixation devices are used in orthopedic surgery to align and/or fix a desired relationship between adjacent vertebral bodies. Such devices typically include a spinal fixation element, such as a relatively rigid fixation rod or plate, that is coupled to adjacent vertebrae by attaching the element to various anchoring devices, such as hooks, bolts, wires, or screws. The fixation elements can have a predetermined contour that has been designed according to the properties of the target implantation site, and once installed, the fixation element holds the vertebrae in a desired spatial relationship, either until desired healing or spinal fusion has taken place, or for some longer period of time.

Spinal fixation elements can be anchored to specific portions of the vertebrae. A variety of anchoring devices have been developed to facilitate engagement of a particular portion of the bone. Pedicle screw assemblies, for example, have a shape and size that is configured to engage pedicle bone. Such screws typically include a threaded shank that is adapted to be threaded into a vertebra, and a head portion having a rod-receiving element, often in the form of a U-shaped recess formed in the head. A set-screw, plug, or similar type of closure mechanism is used to lock the fixation element, e.g., a spinal rod, into the rod-receiving head of the pedicle screw. In use, the shank portion of each screw is threaded into a vertebra, and once properly positioned, a rod is seated through the rod-receiving member of each screw and the rod is locked in place by tightening a cap or other closure mechanism to securely interconnect each screw and the fixation rod.

Recently, the trend in spinal surgery has been moving toward providing minimally invasive devices and methods for implanting bone anchors and spinal fixation devices. One such method, for example, is disclosed in U.S. Pat. No. 6,530,929 of Justis et al. and it utilizes two percutaneous access devices for implanting an anchoring device, such as a spinal screw, into adjacent vertebrae. A spinal rod is then introduced through a third incision a distance apart from the percutaneous access sites, and the rod is transversely moved into the rod-engaging portion of each spinal screw. The percutaneous access devices can then be used to apply closure mechanisms to the rod-engaging heads to lock the rod therein. While this procedure offers advantages over prior art invasive techniques, the transverse introduction of the rod can cause significant damage to surrounding tissue and muscle.

Accordingly, there remains a need for improved minimally invasive devices and methods.

### SUMMARY

Disclosed herein are instruments and methods that facilitate the treatment of spinal disorders in a minimally invasive manner. In particular, the disclosed methods permit the delivery and implanting of one or more bone anchors and/or one or more fixation elements, for example, a spinal rod, in a minimally invasive manner thereby limiting trauma to surrounding tissue. Moreover, certain exemplary methods disclosed herein facilitate the removal of diseased disc material and the placement of bone graft to promote spinal fusion, on one or both sides of the spine, in a minimally invasive manner. Also, disclosed herein are instruments that facilitate the subcutaneous connection of a fixation element, such as a spinal rod, to a bone anchor.

In accordance with one exemplary embodiment, a minimally invasive surgical method may comprise positioning a first anchor and a second anchor in a first vertebra and a second vertebra, respectively, through a first incision made on a first side of a patient's spine, percutaneously positioning a third anchor in a third vertebra through a second incision distinct from the first incision, advancing the first end of a fixation element from the first incision subcutaneously to the third anchor, and coupling the fixation element to the first anchor, the second anchor, and the third anchor.

In accordance with another exemplary embodiment, a minimally invasive surgical method may comprise positioning a first bone screw and a second bone screw into a first pedicle of a first vertebra and a first pedicle of a second vertebra, respectively, through a first incision made on a first side of a patient's spine and percutaneously positioning a third bone screw into a first pedicle of a third vertebra through a second incision. In the exemplary method, the second incision may be located on the first side of the patient's spine and may be distinct from the first incision. The exemplary method may further comprise positioning a fourth screw and a fifth screw into a second pedicle of the second vertebra and a second pedicle of the third vertebra, respectively, through a third incision made on a second side of the patient's spine and percutaneously positioning a sixth bone screw into a second pedicle of the first vertebra through a fourth incision. In the exemplary method, the fourth incision may be located on the second side of the patient's spine and may be distinct from the third incision.

The exemplary method may further comprise positioning the first end of a first spinal rod in the first incision and advancing the first end of the first spinal rod subcutaneously to the third bone screw. In addition, the exemplary method may comprise coupling the spinal rod to the first bone screw, the second bone screw, and the third bone screw.

The exemplary method may further comprise positioning the first end of a second spinal rod into the third incision and advancing the first end of the second spinal rod subcutaneously to the sixth bone screw. In addition, the exemplary method may comprise coupling the second spinal rod to the fourth bone screw, the fifth bone screw, and the sixth bone screw.

In certain exemplary embodiments, the exemplary minimally invasive surgical method may comprise removing disk material from a first disk space between the first and second vertebrae through the first incision and removing disk material from a second disk space between the second and third vertebrae through the third incision. In addition, the exemplary method may comprise inserting bone graft into the first disk space through the first incision and inserting bone graft into the second disk space through the third incision.

### BRIEF DESCRIPTION OF THE DRAWINGS

These and other features and advantages of the methods and instruments disclosed herein will be more fully understood by reference to the following detailed description in conjunction with the attached drawings in which like reference numerals refer to like elements through the different views. The drawings illustrate principles of the methods and instruments disclosed herein and, although not to scale, show relative dimensions.

FIG. 1 is a posterior view of a patient's back, schematically illustrating three adjacent vertebrae and an exemplary method of minimally invasive spine surgery;

FIG. 2 is a side elevational view of one side of a patient's back, schematically illustrating the positioning of a spinal fixation element relative to the three adjacent vertebrae illustrated in FIG. 1;

FIG. 3 is an end view of one of the vertebra of FIG. 1, schematically illustrating the positioning of the spinal fixation element relative to a bone anchor implanted in the vertebra;

FIG. 4 is a perspective view of an exemplary retractor blade, illustrating a slot formed in the retractor blade to facilitate subcutaneous positioning of a spinal fixation element; and

FIG. 5 is a posterior view of a patient's back, schematically illustrating three adjacent vertebrae and another exemplary method of minimally invasive spine surgery.

### DETAILED DESCRIPTION

Certain exemplary embodiments will now be described to provide an overall understanding of the principles of the structure, function, manufacture, and use of the instruments and methods disclosed herein. One or more examples of these embodiments are illustrated in the accompanying drawings. Those of ordinary skill in the art will understand that the instruments and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

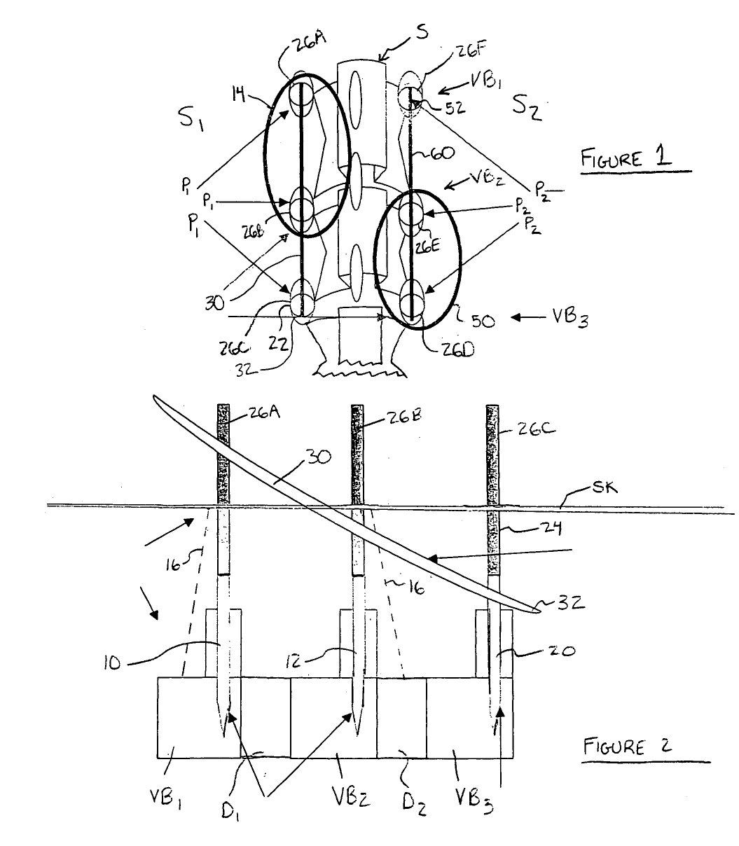

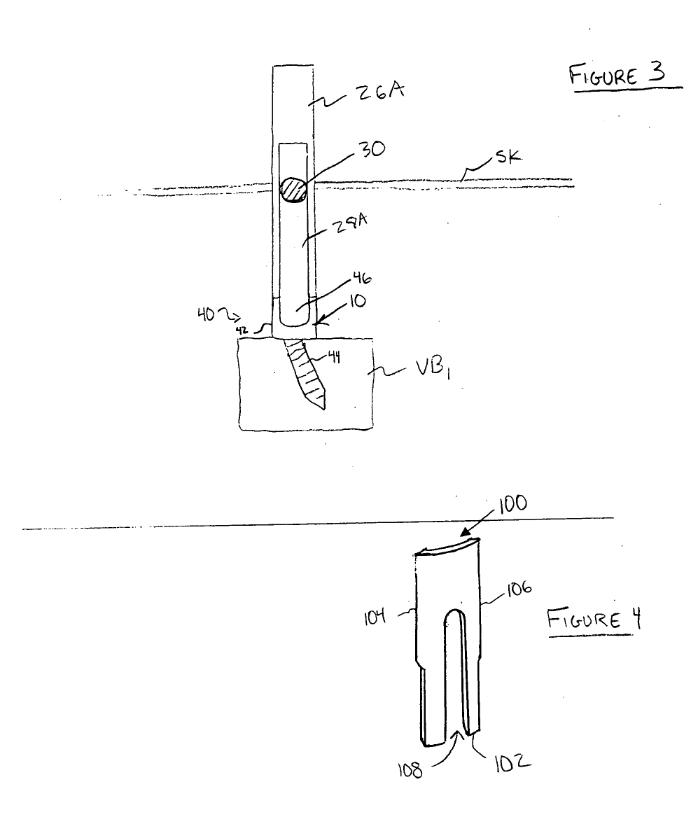

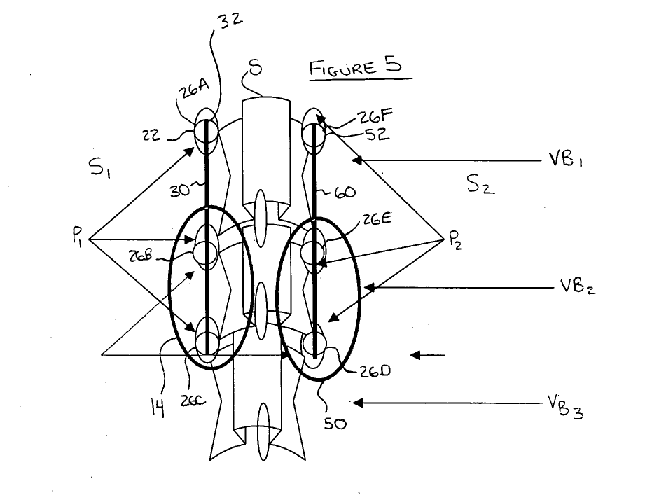

FIGS. 1 and 2 illustrate an exemplary embodiment of a minimally invasive surgical method that provides for the placement of multiple bone anchors and a fixation element on one or both sides of a patient's spine. The exemplary method may be employed to stabilize and align three or more bone segments, in particular, three vertebrae (VB₁, VB₂, VB₃), in a minimally invasive manner that reduces trauma to adjacent tissue. Although the exemplary method described below is designed primarily for use in spinal applications, such as to stabilize and align adjacent vertebrae to facilitate fusion of the vertebrae, one skilled in the art will appreciate that the principles of the exemplary method, as well as the other exemplary embodiments described below, may be applied to any fixation device used to connect multiple sections of bone. Non-limiting examples of applications of the exemplary minimally invasive surgical methods described herein include long bone fracture fixation/stabilization, small bone stabilization, lumbar spine and thoracic stabilization/fusion, cervical spine compression/fixation, and skull fracture/reconstruction plating.

Continuing to refer to FIGS. 1 and 2, the exemplary method may comprise positioning a first anchor 10 and second anchor 12 in a first vertebra VB₁ and a second vertebra VB₂, respectively, through a first skin incision 14 made on a first side S₁ of a patient's spine S. The first and second bone anchors 10, 12 may be any type of conventional bone anchor, including, for example, a monoaxial or polyaxial bone screw, a bolt, or a hook. The first and second bone anchors 10, 12 may be implanted into any portion of the first and second vertebrae VB₁, VB₂, respectively, in any conventional manner through the first incision 14. In the illustrated embodiment, the first bone anchor 10 is implanted into a first pedicle P₁ of the first vertebra VB₁ and the second anchor 12 is implanted in a first pedicle P₁ of the second vertebra VB₂.

The first incision 14 may be a minimally invasive incision made in the patient's skin SK that is expanded, for example, by retraction and or dilation, to create a first pathway 16 from the first incision 14 to the proximate the first vertebra VB₁ and the second vertebra VB₂. FIGS. 1 and 2 illustrate the first incision 14 after expansion, in the lateral, longitudinal, and distal directions, to create the first pathway 16. The location, size, shape, and amount and orientation of expansion of the first incision 14 will depend on the procedure being performed and the number and type of implants being employed. The first and second bone anchors 10, 12, as well as any additional implants employed during the procedure, may be advanced to a respective anchor site proximate the first and second vertebrae VB₁ and VB₂ through the first pathway 16.

The first incision 14 may be expanded to create the first pathway 16 in any conventional manner. In certain embodiments, for example, the first incision 14 may be expanded by dilation to the desired size, shape, and orientation. For example, the first incision may be sequentially dilated using a plurality of dilators to create the first pathway 16. Exemplary methods and instruments for serial dilation are described in commonly owned U.S. Pat. No. 6,159,179, entitled Cannula and Sizing and Insertion Method; U.S. patent application Ser. No. 10/024,221, filed Oct. 30, 2001, entitled Non-Cannulated Dilators; and U.S. patent application Ser. No. 10/021,809, filed Oct. 30, 2001, entitled Configured and Sized Cannulas, each of which are incorporated herein by reference. Once dilation is concluded, a cannula may be inserted into the dilated first incision 14 to define the first passageway 16. Alternatively, a retractor may be inserted into the dilated first incision 12 to further expanded the first incision and/or to define the first pathway 16.

In certain exemplary embodiments, the first incision 14 may be expanded by inserting one or more retractors into the incision and expanding the incision to the desired size, shape, and orientation by expanding the retractor accordingly. The expanded retractor can define the first pathway 16. Any type of conventional retractor or retractors may be employed to expand the first incision 14. For example, suitable retractors are described in commonly owned U.S. patent application Ser. No______, filed Mar. 31, 2004, entitled Telescoping Blade Assemblies and Instruments for Adjusting an Adjustable Blade (Attorney Docket No. DEP5291); U.S. Provisional Patent Application Ser. No. 60/530,655, filed Dec. 18, 2003, entitled Surgical Retractor Systems, Illuminated Cannula and Methods of Use; and U.S. patent application Serial No.______, entitled Surgical Retractor Positioning Device (Attorney Docket No. 3518-1014-000, filed Mar. 25, 2004 each of which are incorporated herein by reference.

In certain exemplary embodiments, the first incision 14 may be expanded to create the first pathway by an intermuscular procedure that includes locating a muscle plane separating two muscles and separating the muscles at the muscle plane to create the first pathway. For example, in certain exemplary methods, the intermuscular plane separating the multifidus and longissimus muscles may be located through the first incision. The multifidus and longissimus muscles may be separated at the muscle plane be inserting a finger or an instrument, such as a retractor, through the muscle plane and advancing the finger or instrument to the vertebra to create the first pathway. Intermuscular procedures are described in detailed in U.S. Pat. No. 6,692,434, entitled Method and Device for Retractor for Microsurgical Intermuscular Lumbar Arthrodesis; U.S. patent application Ser. No. 10/060,905, filed Jan. 29, 2002, entitled Retractor and Method for Spinal Pedicle Screw Placement; and New Uses and Refinements of the Paraspinal Approach to the Lumbar Spine, L. L. Wiltse and C. W. Spencer, Spine, Vol. 13, No. 6, Nov. 6, 1988, each of which is incorporated herein by reference.

Continuing to refer to FIGS. 1 and 2, the exemplary method may further include percutaneously positioning a third anchor 20 in a third vertebra VB₃ through a second incision 22 distinct from the first incision 14. The second incision 22 is preferably a minimally invasive percutaneous skin incision that has a shape and extent that is typically less than or equal to the extent of the instruments and implants being inserted thereto. In certain exemplary embodiments, for example, the second incision 22 may be a stab incision that is expanded to facilitate positioning of the third bone anchor 20 therethrough.

The exemplary method may include creating a second pathway 24 from the percutaneous second incision 22 to the third vertebra VB₃ and advancing the third anchor 20 through the second pathway 24 to the third vertebra VB₃. The second incision 22 may be expanded, e.g., in the lateral, longitudinal, and distal direction, to create the second pathway 24. For example, the second incision 22 may be dilated to the first pedicle P₁ of the third vertebra VB₃ to create the second pathway 24 that extends from the second incision 22 to the first pedicle P₁ of the third vertebra VB₃. The second incision 22 may be dilated by a single dilator, by sequential dilation using multiple dilators, by an expandable retractor, or by other conventional dilation instruments. In certain exemplary embodiments, a cannula may inserted into the dilated second incision 22 to define the second pathway 24.

In certain exemplary embodiments, including the illustrated embodiment, a percutaneous access device 26 may be attached to the third bone anchor 20, as well as the first bone anchor 10 and the second bone anchor 12, to facilitate positioning of the bone anchor and the delivery of implants and instruments to the bone anchor. The percutaneous access device may be sized to span from at least the percutaneous second incision 22 to the third vertebra VB₃ and may have a lumen that defines the second pathway 24 from a proximal end of the percutaneous access device to the third bone anchor 22. FIGS. 1 and 2 illustrate the second incision 22 in an expanded configuration in which the outer diameter of the percutaneous access device 26C defines the perimeter of the expanded second incision 26C and defines the second pathway 24 to proximate the third vertebra VB₃. The percutaneous access devices may have a longitudinal slot formed therein to facilitate positioning of an instrument and/or an implant, such as a spinal rod, relative to the third bone anchor 20. A closure mechanism, e.g., for securing a fixation element to the bone anchor, and/or other components of the third bone anchor 20 may be delivered to the third bone anchor 20 through the lumen of the percutaneous access device 26. Exemplary percutaneous access devices and methods of using such devices are described in commonly owned U.S. patent application Ser. No. 10/738,286, filed Dec. 16, 2003, entitled Percutaneous Access Devices and Bone Anchors; U.S. patent application Ser. No. 10/738,130, filed Dec. 16, 2003, entitled Methods and Devices for Minimally Invasive Spinal Fixation Element Placement; and U.S. patent application Ser. No. 10/737,537, filed Dec. 16, 2003, entitled Method and Device for Spinal Fixation Element Placement, each of which are incorporated herein by reference. In the illustrated embodiment, a percutaneous access device 26 is illustrated connected to each of the bone anchors. One skilled in the art will appreciate that the use of such a percutaneous access device is optional and that in other exemplary embodiments, one, some, or all of the bone anchors may be provided with such a percutaneous access device.

Continuing to refer to FIGS. 1 and 2, the exemplary method may further include advancing the first end 32 of a fixation element 30, such as a spinal rod, e.g., in the illustrated embodiment, or a plate, from the first incision 14 subcutaneously to the third anchor 20 and coupling the fixation element 30 to the first anchor 10, the second anchor 12, and the third anchor 20. In the illustrated embodiment, for example, the first end 32 of the fixation element 30 may be inserted through the first incision 14 into the first pathway 16. The first end 32 may then be advanced from the first pathway 16, subcutaneously, i.e., beneath the skin SK, and preferably, subfascially, i.e., beneath the fascia, to the third anchor 20, as illustrated in FIG. 2. The first end 32 of the spinal fixation element 30 may shaped to facilitate subcutaneous positioning of the fixation element 30. For example, the first end 32 may have a bullet-shaped tip.

In embodiments employing a percutaneous access device 26C coupled to the third anchor 20, the first end 32 of the fixation element 30 may be advanced into an opening, such as a longitudinal slot, provided in the percutaneous access device and advanced distally within the slot to seat the fixation element 30 in the third anchor 20. The position of the fixation element 30 during advancement to the third anchor 20 may be monitored by fluoroscopy or other imaging techniques. In certain exemplary embodiments, including the illustrated embodiment, the percutaneous access devices 26A, 26B, & 26C coupled to each of the bone anchors 10, 12, & 20, respectively, may facilitate subcutaneous advancement of the fixation element 30. For example, each of the percutaneous access devices 26A, 26B, & 26C may include a slot or opening, as discussed above, that may be used to guide the fixation element 30 during subcutaneous advancement. In certain exemplary embodiments, each percutaneous access device 26A, 26B, & 26C may have a longitudinally extending slot 28 that extends distally from the bone anchor that may be used to guide the fixation element 30. In the illustrated embodiment, for example, each percutaneous access device 26A-C has a slot 28 that extends proximally from the bone anchor to above the skin level, as illustrated schematically in FIG. 3. The exemplary method may include aligning the longitudinal slots 28 of each percutaneous access device 26A-C, placing the fixation element through the slots 28 in the first and second percutaneous access device 26A and 26B, and advancing the first end 32 of the fixation element 30 to the slot 28 in the third percutaneous access device 26C. The slots 28 of the percutaneous access device 26 may be aligned with an instrument, including e.g., another fixation element, that extends between each of the slots 28. Such an instrument may be positioned in at the proximal end of each slot 28, preferably above the skin level.

In certain exemplary embodiments, including the illustrated embodiment, the each bone anchor 10, 12, 20 may be a polyaxial screw assembly 40 having a head 42 for receiving the fixation element, e.g., a spinal rod 30, and a bone screw 44 having a threaded shaft that is configured to engage bone, as illustrated in FIG. 3. The head 42 may have a slot 46 for receiving the spinal rod 30 and may be configured to engage a percutaneous access device 26 such that the slot 46 in the head 42 of the polyaxial screw assembly 40 is aligned with the slot 28 in the percutaneous access device 26. Alignment of the slots 26, 46, facilitates placement of the spinal rod 30 relative to the polyaxial screw assembly 40.

The exemplary method may further include positioning the fixation element 30 relative to the first and second anchors 10, 12. For example, the fixation element 30 may be seated in a portion of each of the bone anchors 10, 12. Once positioned, the fixation element 30 may be coupled to each of the bone anchors 10, 12, 20, by, for example, a closure mechanism secured to each of the bone anchors.

Once implanted, the fixation element 30 may be fixed at opposing ends to the first and third bone anchors 10, 20 and centrally at the second bone anchor 12. In this manner, the fixation element 30 spans three adjacent vertebrae (VB₁, VB₂ VB₃) to fix the adjacent vertebrae relative to one another.

Continuing to refer to FIGS. 1 and 2, the exemplary method may include removing the disk material between two adjacent vertebrae to facilitate fusion of the vertebrae to one another. For example, the first incision 14 and the first pathway 16 may provide access to the disk space D₁ between the first vertebra VB₁ and the second VB₂. Disk material may be removed from the disk space D₁ in any conventional manner. Upon removal of the disk material from the disk space D₁, bone graft or other bone fusion promoting material, such as bone morphogenic proteins (BMPs), may be positioned within the disc space D₁ to promote fusion of the first vertebra VB₁ to the second vertebra VB₂. In certain exemplary embodiments, an interbody fusion device, such as a cage, may be packed with bone graft or other bone fusion promoting materials and positioned in the disk space D₁ between the vertebra. In certain exemplary embodiments, the removal of disk material and placement of bone graft may be conducted in accordance with a transforaminal interbody fusion (TLIF) procedure.

One skilled in the art will appreciate that the order of the steps of the exemplary method described above may be varied without departing from the scope of the present invention. For example, the order of making the incisions and placement of the both anchors may be varied and/or the interbody fusion procedures may be performed before anchor placement or positioning of the fixation element. Moreover, one skilled in the will appreciate that the exemplary method may be performed on more than three vertebrae and/or on two or more non-adjacent vertebrae.

The exemplary method may further comprise making a third incision 50 on the other, second side S₂ of the patient's spine to provide access to one or more vertebrae. In the illustrated embodiment, for example, the third incision 50 provides access to the second vertebra VB₂ and the third vertebra VB₃, as well as the disk space D₂ therebetween, as illustrated in FIG. 1. The third incision 50 may be analogous in size and shape to the first incision 14, described above. For example, the third incision 50 may be expanded to provide a pathway from the third incision 50 the second and third vertebrae VB₂, VB₃.

In certain exemplary embodiments, including the illustrated embodiment, the exemplary method may include removing disk material from the disk space D₂ between the second and third vertebrae VB₂, VB₃ through the third incision 50. Disk material may be removed in any conventional manner using convention instruments. In addition, the exemplary method may include inserting bone graft and/or other bone fusion promoting material into the disk space D₂ between the second and third vertebrae VB₂, VB₃ through the third incision 50. The bone graft and/or other bone fusion promoting material may be positioned within the disc space D₂ to promote fusion of the second vertebra VB₂ to the third vertebra VB₃. In certain exemplary embodiments, an interbody fusion device, such as a cage, may be packed with bone graft or other bone fusion promoting materials and positioned in the disk space D₂ between the second vertebra VB₂ and the third vertebra VB₃. In certain exemplary embodiments, the removal of disk material and placement of bone graft may be conducted in accordance with a transforaminal interbody fusion (TLIF) procedure. In such exemplary embodiments, a two-level fusion procedure may be performed using minimally invasive skin incisions (e.g., the first incision, the second incision, and the third incision) on opposite sides of the spine.

In certain exemplary embodiments, including the illustrated embodiment, it may be desirable to fix vertebrae to be fused on both sides of the spine, although, as one skilled in the will appreciate, it may be desirable in other cases to fix the vertebrae on only one side of the spine. Accordingly, the exemplary method may include positioning a fourth anchor and a fifth anchor in the second and third vertebrae VB₂, VB₃, respectively, through the third incision 30. For example, the fourth anchor may be implanted into the second pedicle P2 of the second vertebrae VB₂ and the fifth anchor may be implanted into the second pedicle P2 of the third vertebrae VB₃. A fixation element, such as a spinal rod, may coupled to the fourth and fifth anchor to fix the second and third vertebrae VB₂, VB₃ on both sides S₁, S₂ of the spine.

Continuing to refer to FIG. 1, the exemplary embodiment may include percutaneously positioning a sixth bone anchor in the first vertebra VB₁ through a fourth, percutaneous incision 52 that is distinct from the third incision 52 and located on the second side S₂ of the patient's spine. The fourth incision 42 may be expanded, e.g., in the lateral, longitudinal, and distal direction, to create a percutaneous pathway from the fourth incision 52 to an anchor site on the first vertebra VB₁. For example, the fourth incision 52 may be dilated to the second pedicle P₂ of the first vertebra VB₁ to create a percutaneous pathway that extends from the fourth incision 52 to the second pedicle P₂ of the first vertebra VB₁. The fourth bone anchor may be implanted in the second pedicle P₂ of the first vertebra VB or in any other portion of the first vertebra VB₁. The fourth incision 52 may be analogous to the second incision 22 described above. For example, the fourth incision 52 may be dilated by a single dilator, by sequential dilation using multiple dilators, by an expandable retractor, or by other conventional dilation instruments. In certain exemplary embodiments, a cannula may inserted into the dilated fourth incision 52 to define the pathway to the first vertebra VB₁.

Continuing to refer to FIG. 1, the exemplary method may further include advancing the first end of a second fixation element 60, such as a spinal rod, e.g., in the illustrated embodiment, or a plate, from the third incision 50 subcutaneously to the sixth anchor and coupling the second fixation element 60 to the fourth anchor, the fifth anchor, and the sixth anchor. In the illustrated embodiment, for example, the first end of the second fixation element 60 may be inserted through the third incision 50 into the pathway extending to the second and third vertebra VB₂ and VB₃. The first end of the second fixation element may then be advanced from the pathway, subcutaneously, i.e., beneath the skin SK, and preferably, subfascially, i.e., beneath the fascia, to the sixth anchor. The first end of the second spinal fixation element 60 may be shaped to facilitate subcutaneous positioning of the fixation element. For example, the first end may have a bullet-shaped tip. Once positioned, the second fixation element 60 may be coupled to each of the bone anchors by, for example, a closure mechanism secured to each of the bone anchors.

In certain exemplary embodiments, including the illustrated embodiment, a percutaneous access device 26F may be attached to the sixth bone anchor, as well as the fourth bone anchor (percutaneous access device 26E) and the fifth bone anchor (percutaneous access device 26D), to facilitate positioning of the bone anchor and the delivery of implants and instruments to the bone anchor. The percutaneous access device(s) may be constructed in a manner analogous to the percutaneous access devices discussed above.

One skilled in the art will appreciate that the order of steps of the exemplary method may be varied without departing from the scope of the present invention. The order of steps set forth above is merely exemplary, and is not intended to the limit the methods of the claimed invention.

Referring to FIG. 5, another exemplary embodiment of a method of minimally invasive surgical method may include inserting the second and third anchors through a first incision 14 that provides access to the second and third vertebrae VB₂, VB₃. In the illustrated method, the first bone anchor is delivered to the first vertebra VB1 through a percutaneous, second incision 22. The disk space D2 between the second and third vertebrae VB₂, VB₃ may be accessed through the first incision 14 or the third incision 50 to for example, remove the disk material and position bone graft and an interbody disk material into the disk space D₂. A fixation element 30 may be inserted through the first incision 14 to connect the fixation element 30 to the first, second, and third vertebrae, VB₁₋₃, on the first side S₁ of the spine S, in the manner described above. A second fixation element 60 may be inserted through the third incision 50 to connect the second fixation element 60 to the first, second, and third vertebrae, VB₁₋₃, on the second side S₂ of the spine S, in the manner described above.

In certain exemplary embodiments, multiple spinal fixation elements may be employed to fix two or more vertebrae on one side of the spine. For example, referring to FIG. 5, one spinal fixation element may connect the first and second vertebrae VB1, VB2 and a second spinal fixation may connect the second and third vertebrae VB2, VB3. The two spinal fixation elements may be similarly or dissimilarly constructed. For example, in certain exemplary embodiments, one fixation element may provide a rigid connection between two adjacent vertebrae and another fixation element may provide a dynamic connection between two adjacent vertebrae.

FIG. 3 illustrates an exemplary embodiment of a retractor blade 100 having a longitudinal slot 108 extending proximally from the distal end 102 of the retractor blade 100. The slot 108 may be configured, e.g. sized, shaped, and oriented, to facilitate passage of a spinal rod therethrough and alignment of the spinal rod during subcutaneous positioning of the rod. For example, the retractor blade 100 may be employed in a retractor positioned in an incision, such the first, second, third, and/or fourth incision, to allow a rod, or other spinal fixation element, to be subcutaneously positioned from within the working channel of the retractor through the slot 108 in the retractor blade 100 to a desired site outside of the retractor working channel. In the illustrated embodiment, the slot 108 is centrally located between two opposing side walls 104, 106 of the retractor blade 100, although the slot may be positioned at other locations and need not be oriented longitudinally. The slot 108 may be sized to allow a spinal rod to pass therethrough. For example, the width of the slot 108 is preferably less than the diameter of the rod selected to pass therethrough.

The retractor blade 100 may be a telescoping blade that is adjustable longitudinally relative to a fixed blade or may be a fixed blade. The retractor blade 100 may be used alone, used in combinations with other similar or dissimilar retractor blades, or coupled to a retractor or other instrument.

While methods and instruments of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

1. A minimally invasive surgical method comprising:
making a first incision in a patient;
expanding the first incision to create a first pathway from the first incision to a first vertebra and a second vertebra;
advancing a first anchor through the pathway to a first anchor site on the first vertebra;
advancing a second anchor through the pathway to a second anchor site on the second vertebra;
making a percutaneous incision in the patent;
advancing a third anchor through the percutaneous incision to a third anchor site on the third vertebra;
positioning a first end of a fixation element in the first pathway;
advancing the first end of the fixation element subcutaneously to the third anchor; and
coupling the fixation element to the first anchor, the second anchor, and the third anchor.

2. The method of claim 1, wherein expanding the first incision includes dilating the first incision to the first and second vertebrae.

3. The method of claim 2, wherein dilating the first incision comprises sequentially dilating the first incision to the first and second vertebrae.

4. The method of claim 2, wherein expanding the first incision further includes inserting a cannula into the dilated first incision, the cannula defining the first pathway from the first incision to the first and second vertebrae.

5. The method of claim 2, wherein expanding the first incision further includes inserting a retractor into the dilated first incision and expanding the retractor within the first incision, the retractor the defining the first pathway from the first incision to the first and second vertebrae.

6. The method of claim 5, wherein the retractor includes a retractor blade having an opening formed therein that is configured to allow the first end of the fixation element to pass therethrough.

7. The method of claim 1, wherein expanding the first incision comprises inserting a retractor into the first incision and expanding the retractor within the first incision, the retractor the defining the first pathway from the first incision to the first and second vertebrae.

8. The method of claim 1, further comprising creating a second pathway from the percutaneous incision to the third vertebra and advancing the third anchor through the second pathway to the third anchor site.

9. The method of claim 8, wherein creating a second pathway comprises dilating the percutaneous incision to the third vertebra and inserting a cannula into the dilated percutaneous incision, the cannula defining the second pathway from the percutaneous incision to the third vertebra.

10. The method of claim 1, wherein the third anchor has a percutaneous access device attached thereto, the percutaneous access device being sized to span from at least the percutaneous incision to the third vertebra, the percutaneous access device having a lumen that defines a second pathway from a proximal end of the percutaneous access device to the third bone anchor.

11. The method of claim 10, wherein the percutaneous access device has an opening formed therein to facilitate coupling of the fixation element to the third bone anchor.

12. The method of claim 10, further comprising advancing a closure mechanism through the lumen of the percutaneous access device and engaging the closure mechanism to the third bone anchor to couple the fixation element to the third bone anchor.

13. The method of claim 1, wherein the first, second, and third bone anchors are polyaxial bone screws.

14. The method of claim 1, wherein the fixation element is a spinal rod.

15. The method of claim 14, wherein the first end of the spinal rod has a bullet-shaped tip to facilitate passage of the tip through tissue.

16. The method of claim 1, wherein the first end of the fixation element is advanced subfascially to the third anchor.

17. The method of claim 1, further comprising removing disk material from the disk space between the first and second vertebrae through the first pathway.

18. The method of claim 17, further comprising inserting bone graft into the disk space.

19. The method of claim 18, further comprising inserting an interbody fusion device into the disk space.

20. A minimally invasive surgical method comprising:
positioning a first anchor and second anchor in a first vertebra and a second vertebra, respectively, through a first incision made on a first side of a patient's spine;
percutaneously positioning a third anchor in a third vertebra through an incision distinct from the first incision;
advancing the first end of a fixation element subcutaneously from the first incision to the third anchor; and
coupling the fixation element to the first anchor, the second anchor, and the third anchor.

21. The method of claim 20, wherein the first anchor is adjacent the second anchor.

22. The method of claim 21, wherein the third anchor is adjacent one of the second anchor and the first anchor.

23. The method of claim 20, further comprising removing disk material from the disk space between the first and second vertebrae through the first incision.

24. The method of claim 23, further comprising inserting bone graft into the disk space.

25. The method of claim 24, further comprising inserting an interbody fusion device into the disk space.

26. The method of claim 21, further comprising making a third incision on a second side of the patient's spine opposite the first side of the patient's spine and removing disk material from the disk space between the second vertebra and the third vertebra through the third incision.

27. The method of claim 26, further comprising inserting bone graft into the disk space between the second vertebra and the third vertebra.

28. The method of claim 27, further comprising positioning a fourth anchor and a fifth anchor in the second vertebra and third vertebra, respectively, through the third incision.

29. The method of claim 28, further comprising percutaneously positioning a sixth anchor in the first vertebra through an fourth incision on the second side of the patient's spine, the fourth incision being distinct from the third incision;
positioning the first end of a second fixation element into the third incision;
advancing the first end of the second fixation element subcutaneously to the sixth anchor; and
coupling the second fixation element to the fourth anchor, the fifth anchor, and the sixth anchor.

30. A minimally invasive surgical method comprising:
positioning a first bone screw and a second bone screw into a first pedicle of a first vertebra and a first pedicle of a second vertebra, respectively, through a first incision made on a first side of a patient's spine;
percutaneously positioning a third bone screw into a first pedicle of a third vertebra through a second incision, wherein the second incision is located on the first side of the patient's spine and is distinct from the first incision;
positioning a fourth screw and a fifth screw into a second pedicle of the second vertebra and a second pedicle of the third vertebra, respectively, through a third incision made on a second side of the patient's spine;
percutaneously positioning a sixth bone screw into a second pedicle of the first vertebra through a fourth incision, wherein the fourth incision is located on the second side of the patient's spine and is distinct from the third incision;
positioning the first end of a first spinal rod in the first incision;
advancing the first end of the first spinal rod subcutaneously to the third bone screw;
coupling the spinal rod to the first bone screw, the second bone screw, and the third bone screw;
positioning the first end of a second spinal rod into the third incision;
advancing the first end of the second spinal rod subcutaneously to the sixth bone screw; and
coupling the second spinal rod to the fourth bone screw, the fifth bone screw, and the sixth bone screw.

31. The method of claim 30, further comprising removing disk material from a first disk space between the first and second vertebrae through the first incision and removing disk material from a second disk space between the second and third vertebrae through the third incision.

32. The method of claim 31, further comprising inserting bone graft into the first disk space through the first incision and inserting bone graft into the second disk space through the third incision.

33. The method of claim 32, further comprising inserting an interbody fusion device into the first disk space and inserting an interbody fusion device into the second disk space.

34. A retractor blade comprising:
a body having a proximal end and a distal end, the body having a longitudinally oriented slot extending proximally from the distal end of the body.

35. The retractor blade of claim 34, wherein the slot is centrally located between opposing side walls of the body.

36. The retractor blade of claim 34, wherein the slot is sized to allow a spinal rod to pass therethrough.
